(19) United States
(12) Patent Application Publication
(10) Pub. No.: US 2024/0351979 A1
(43) Pub. Date: Oct. 24, 2024

(54) POLYONIUM SALT PHOTOACID GENERATOR FOR ARF LIGHT SOURCE DRY LITHOGRAPHY, PREPARATION METHOD THEREFOR AND APPLICATION THEREOF

(71) Applicants: SHANGHAI SINYANG SEMICONDUCTOR MATERIALS CO., LTD., Shanghai (CN); CHINA ADVANCED LITHOGRAPHIC MATERIAL TECHNOLOGY CO., LTD., Shanghai (CN)

(72) Inventors: Shunong FANG, Shanghai (CN); Su WANG, Shanghai (CN); Zhiyue GENG, Shanghai (CN)

(21) Appl. No.: 18/685,794

(22) PCT Filed: Oct. 19, 2021
(86) PCT No.: PCT/CN2021/124604

§ 371 Date: Feb. 22, 2024

(30) Foreign Application Priority Data
Aug. 25, 2021 (CN) 202110979286.2

(51) Int. Cl. C07C 381/12; C07C 303/02; C07C 303/22; C07C 309/12; G03F 7/004

(52) U.S. Cl. CPC C07C 381/12; C07C 303/02; C07C 303/22; C07C 309/12; G03F 7/0045

(57) ABSTRACT

Disclosed in the present invention are a polyonium salt photoacid generator for ArF light source dry lithography, and a preparation method therefor and an application thereof. The photoacid generator is an onium salt having an anions and an onium ion, the anion having a structure as shown in formula (I), the onium ion having a structure shown in formula (A) or formula (B), and the number of onium ions keeping the charge of the onium salt neutral. A photoresist comprising the onium salt of the present invention has better resolution, sensitivity and linewidth roughness.

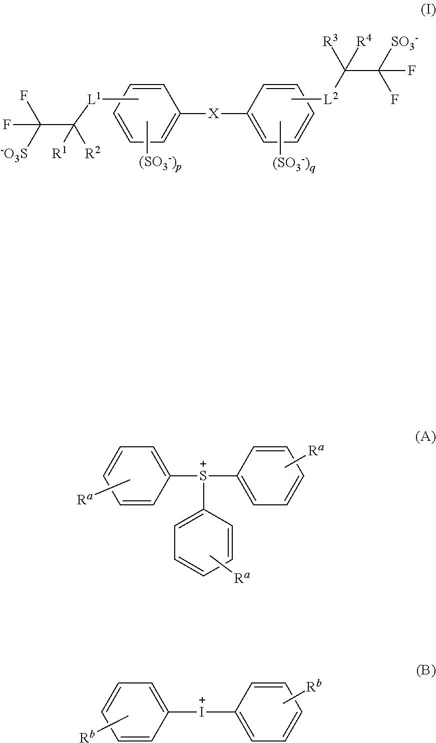

The present application claims the priority of Chinese patent application No. 2021109792862, the application date of which is Aug. 25, 2021. This Chinese patent application is incorporated herein by reference in its entirety.

### TECHNICAL FIELD

The disclosure relates to a polyonium salt photoacid generator for dry lithography by an ArF light source, and a preparation method therefor and the use thereof.

### BACKGROUND

Lithography refers to a pattern micromachining technique that transfers a pattern designed on a mask onto a substrate by means of exposure, development, etching and other processes using the chemical sensitivity of a lithographic material (specifically a photoresist) under the action of visible light, ultraviolet light, electron beams, etc. A lithographic material (specifically a photoresist), also known as a photoresist, is the most critical functional chemical material involved in lithography, the main components thereof being a resin, a photo acid generator (PAG), and corresponding additives and solvents. A photoacid generator is a photosensitive compound, which is decomposed under the illumination to produce an acid, and the acid thus produced can allow a decomposition or cross-linking reaction of an acid-sensitive resin, thereby increasing the dissolution contrast between an illuminated portion and a non-illuminated portion in a developing solution and being applicable to the technical field of pattern micromachining.

The three important parameters of a photoresist, including resolution, sensitivity and line width roughness, determine the process window of the photoresist during chip manufacturing. With the continuous improvement of the performance of semiconductor chips, the integration level of integrated circuits increases exponentially, and patterns in integrated circuits become smaller and smaller. In order to fabricate smaller patterns, it is necessary to improve the above-mentioned three performance indexes of a photoresist. According to the Rayleigh equation, the resolution of a photoresist can be improved by using a short-wavelength light source in a photolithography process. The wavelength of a light source used in a photolithography process has developed from 365 nm (I-line) to 248 nm (KrF), 193 nm (ArF) and 13 nm (EUV). In order to improve the sensitivity of a photoresist, a chemically amplified photosensitive resin is currently used in mainstream KrF, ArF and EUV photoresists. Therefore, a photoacid generator matched with a chemically amplified photosensitive resin is widely used in high-end photoresists.

With the gradual development of a photolithography process to a 193-nm dry process, the complexity of the process increases, and there are increasingly higher requirements on a photoacid generator. It has become an urgent problem to be solved in the industry to develop a photoacid generator that can improve the resolution, sensitivity and line width roughness of a photoresist.

### CONTENT OF THE PRESENT INVENTION

Given the above problems in the prior art, the disclosure aims to provide a brand-new onium salt, which can be used as a photoacid generator to improve various properties of a photoresist, such as the resolution, sensitivity and line width roughness thereof.

The present disclosure provides an onium salt, which has an anion and an onium ion, wherein the anion has a structure represented by formula (I), the onium ion has a structure represented by formula (A) or formula (B), and the number of the onium ions keeps the charge of the onium salt neutral;

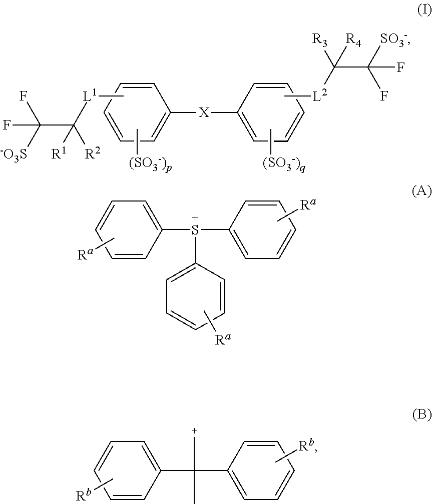

where R¹, R², R³ and R⁴ are each independently H or F;
L¹ and L² are

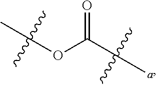 [SMILES: *OC(*)=O]

where the terminal a is connected to a benzene ring;
p and q are each independently 0, 1, 2, 3 or 4;
X is

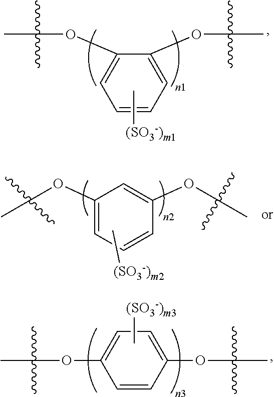

where
n1, n2 and n3 are each independently 1, 2, 3, 4 or 5;
m1, m2 and m3 are each independently 0, 1, 2, 3 or 4; and
R^{a} and R^{b} are each independently a halogen, a C₁₋₂₀ alkyl group or a C₁₋₂₀ alkoxy group; and the numbers of R^{a} and R^{b} are each independently 0 to 5.

In one solution of the present disclosure, R¹, R², R³ and R⁴ may be the same.

In one solution of the present disclosure, R¹, R², R³ and R⁴ may be H.

In one solution of the present disclosure, R¹, R², R³ and R⁴ may be F.

In one solution of the present disclosure, p and q may be the same.

In one solution of the present disclosure, p and q may be 0.

In one solution of the present disclosure, p and q may be 1.

In one solution of the present t disclosure, the structural moiety

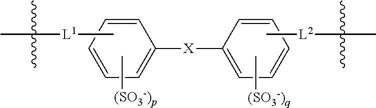

may be

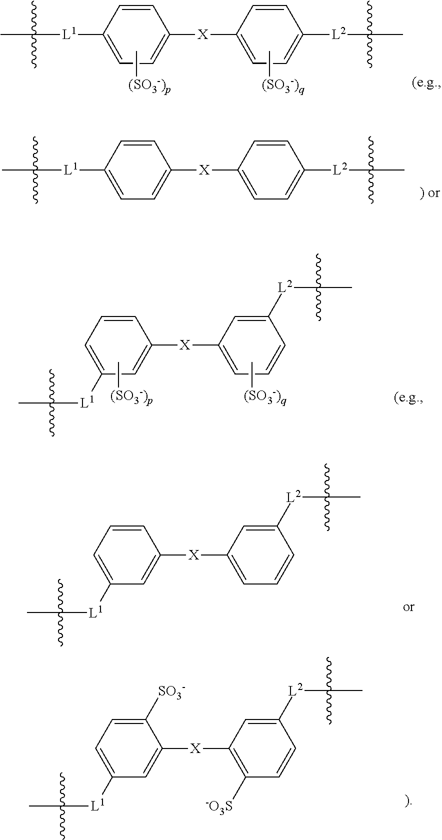

In one solution of the present disclosure, n1 in

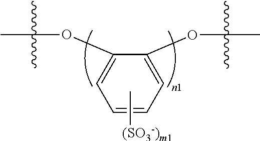

may be 2.

In one solution of the present disclosure, m1 in

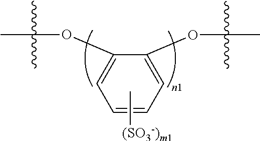

may be 1; for example,

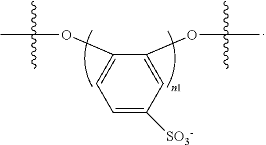

In one solution of the present disclosure,

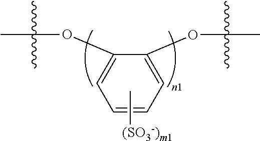

may be

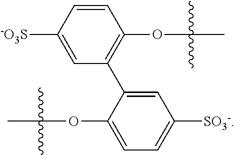

In one solution of the present disclosure, n2 in

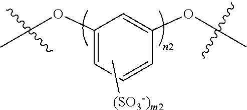

may be 1.

In one solution of the present disclosure, m2 in

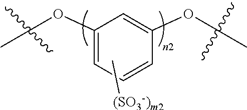

may be 0 or 2; for example,

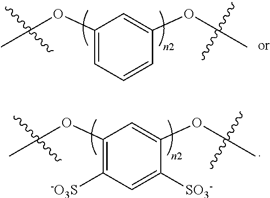

In one solution of the present disclosure,

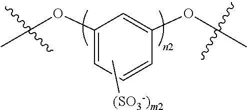

may be

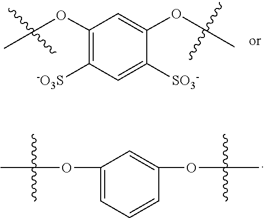

In one solution of the present disclosure, n3 in

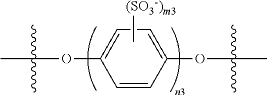

may be 1 or 2.

In one solution of the present disclosure, m3 in

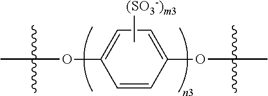

may be 1; for example,

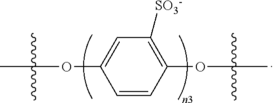

In one solution of the present disclosure,

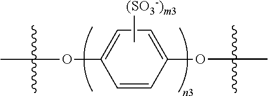

may be

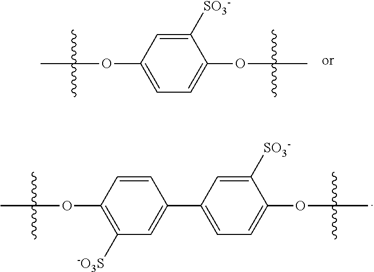

In one solution of the present disclosure, the anion may have a structure represented by formula (I-1)

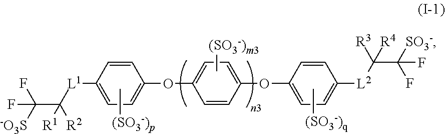

where the definitions of the various groups are as described in any solution of the present disclosure.

In one solution of the present disclosure, the anion may have a structure represented by formula (I-2)

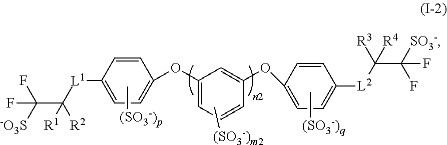

where the definitions of the various groups are as described in any solution of the present disclosure.

In one solution of the present disclosure, the anion may have a structure represented by formula (I-3)

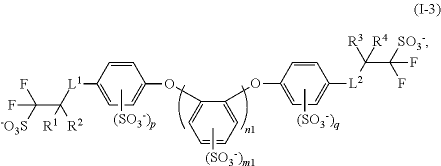

where the definitions of the various groups are as described in any solution of the present disclosure.

In one solution of the present disclosure, the anion may have a structure represented by formula (I-4)

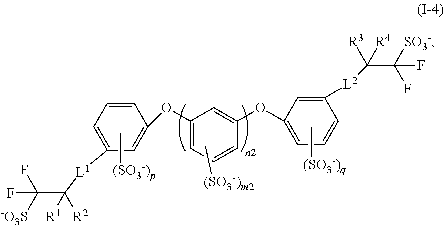

where the definitions of the various groups are as described in any solution of the present disclosure.

In one solution of the present disclosure, the anion may be any one of the following structures:

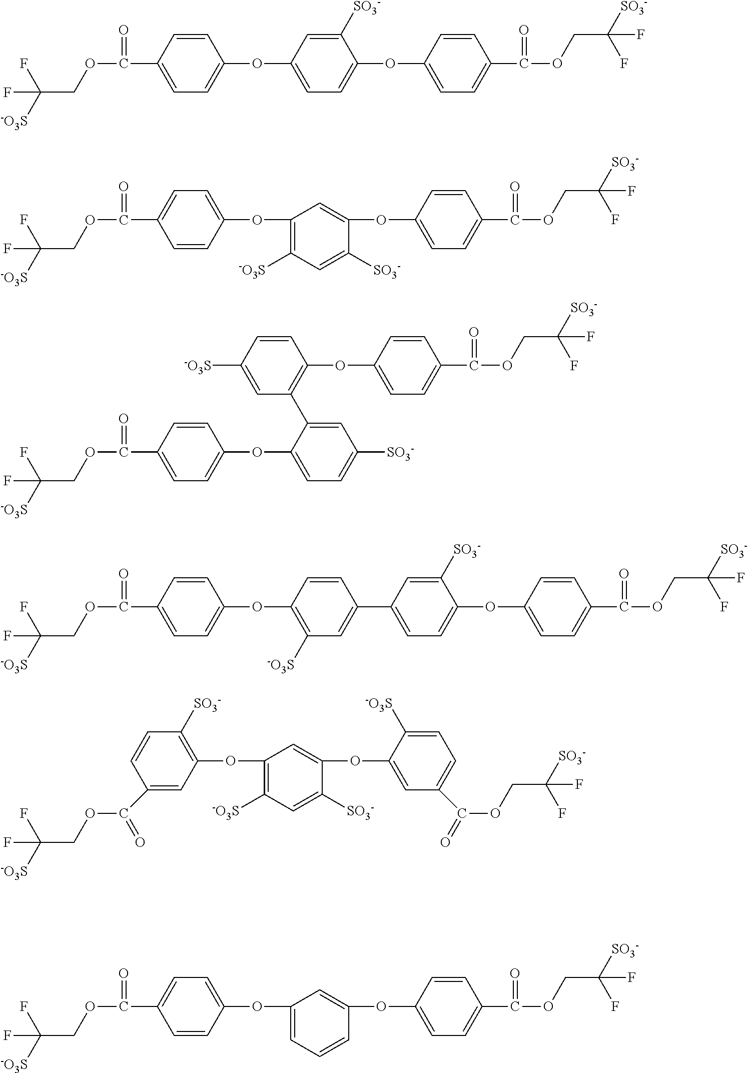

In one solution of the present disclosure, in the definitions of R^{a} and R^{b}, the halogens may each independently be fluorine, chlorine, bromine or iodine. The C₁₋₂₀ alkyl groups may each independently be C₁₋₄ alkyl groups (such as a methyl group, an ethyl group, a propyl group, an isopropyl group, an n-butyl group, an isobutyl group or a tert-butyl group). The C₁₋₂₀ alkoxy groups may each independently be C₁₋₄ alkoxy groups (such as a methoxy group, an ethoxy group, a propoxy group, an isopropoxy group, an n-butoxy group, an iso-butoxy group or a tert-butoxy group).

In one solution of the present disclosure, the structure represented by formula (A) in the onium ion may be:

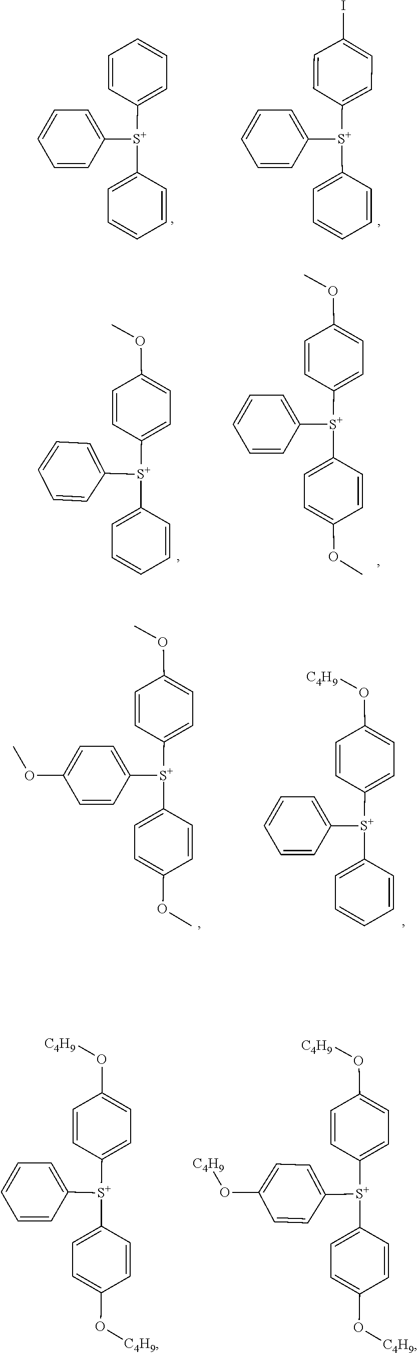

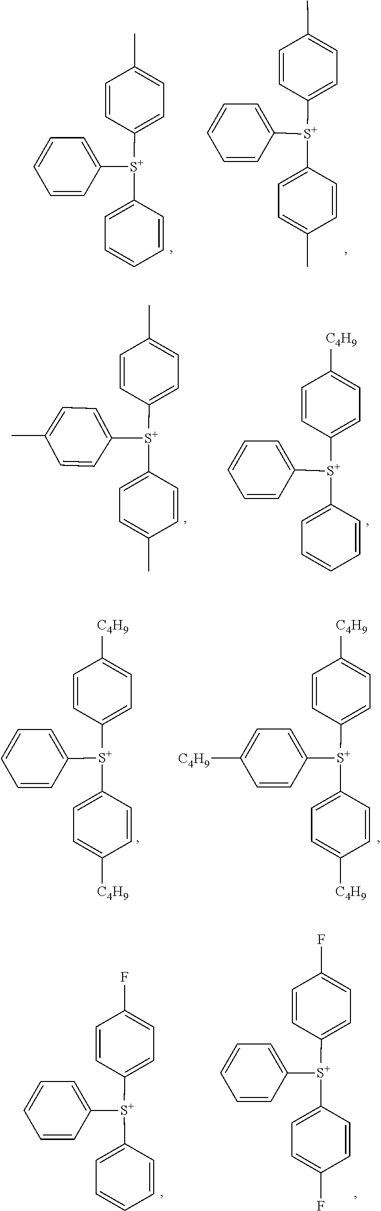

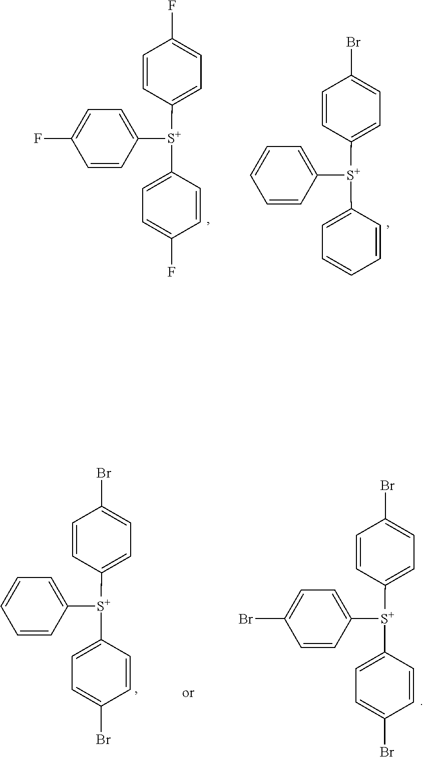 [SMILES: Fc1ccc([S+](c2ccc(F)cc2)c2ccc(F)cc2)cc1 ; Brc1ccc([S+](c2ccccc2)c2ccccc2)cc1 ; Brc1ccc([S+](c2ccccc2)c2ccc(Br)cc2)cc1 ; Brc1ccc([S+](c2ccc(Br)cc2)c2ccc(Br)cc2)cc1]

In one solution of the present disclosure, the structure represented by formula (B) in the onium ion may be:

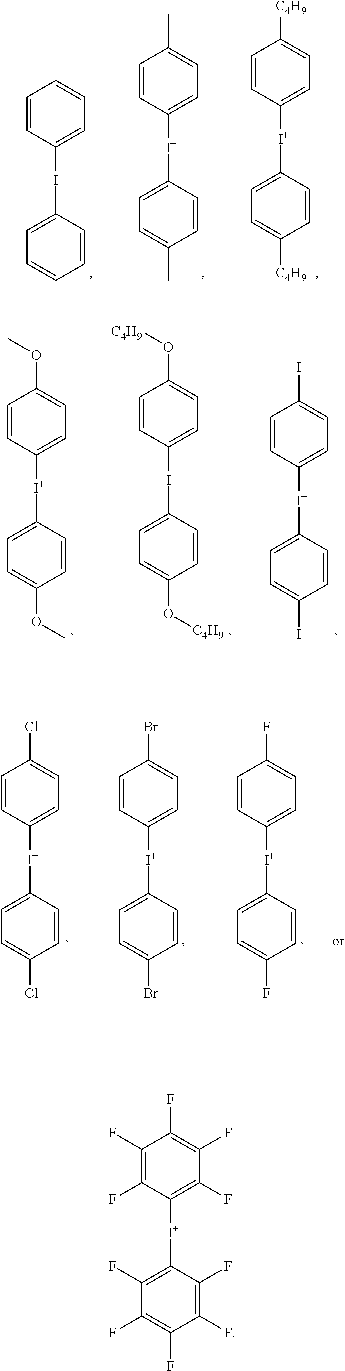

In one solution of the present disclosure, the onium ion may be

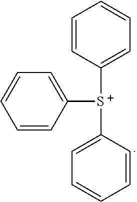 [SMILES: c1ccc([S+](c2ccccc2)c2ccccc2)cc1]

In one solution of the present disclosure, the onium salt may be any one of the following solutions:
solution 1: the anion is

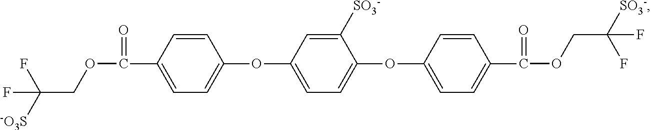

the onium ion is

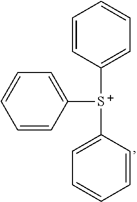 [SMILES: c1ccc([S+](c2ccccc2)c2ccccc2)cc1]

and the number of the onium ions is 3;
solution 2: the anion is

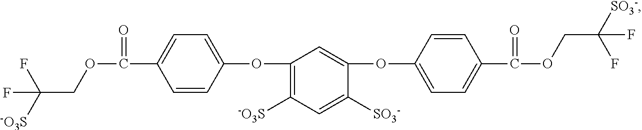

the onium ion is

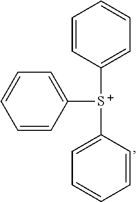 [SMILES: c1ccc([S+](c2ccccc2)c2ccccc2)cc1]

and the number of the onium ions is 4;
solution 3: the anion is

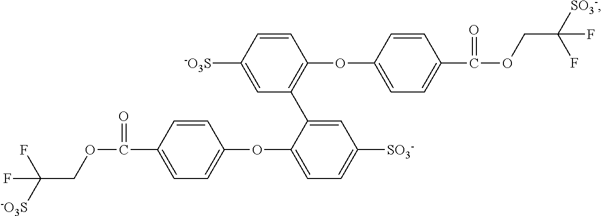

the onium ion is

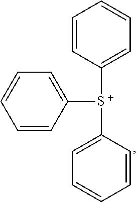 [SMILES: c1ccc([S+](c2ccccc2)c2ccccc2)cc1]

and the number of the onium ions is 4;
solution 4: the anion is

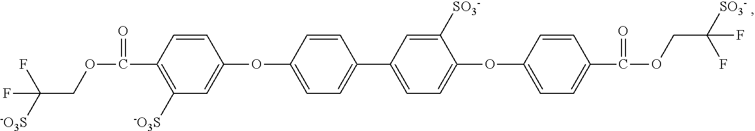

the onium ion is

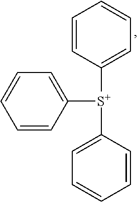 [SMILES: c1ccc([S+](c2ccccc2)c2ccccc2)cc1]

and the number of the onium ions is 4;
solution 5: the anion is

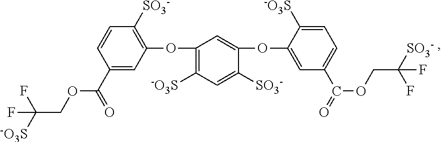

the onium ion is

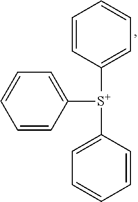 [SMILES: c1ccc([S+](c2ccccc2)c2ccccc2)cc1]

and the number of the onium ions is 6;
solution 6: the anion is

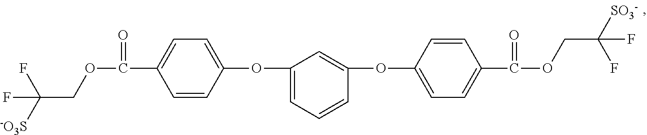

the onium ion is

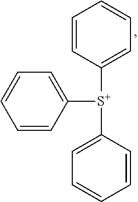 [SMILES: c1ccc([S+](c2ccccc2)c2ccccc2)cc1]

and the number of the onium ions is 2.

The present disclosure further provides a method for preparing the above-mentioned onium salt, the method comprising the following steps: in a solvent, subjecting a compound represented by formula (II) and a compound represented by formula (A-1) or a compound represented by formula (B-1) to a salification reaction, so as to obtain the onium salt;

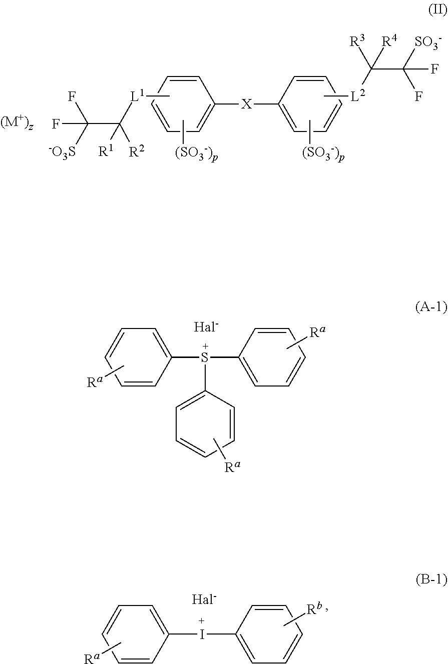

where M⁺ is Li⁺, Na⁺ or K⁺;
Hal⁻ is F⁻, Cl⁻, Br or I⁻;
z is (p+q+2+n1×m1), (p+q+2+n2×m2) or (p+q+2+n3×m3); and

the definition of each of the remaining groups is as described in any solution of the present disclosure.

In the above-mentioned method for preparing an onium salt, the reaction conditions and operation of the salification reaction and the types and amounts of the reagents may be conventional choices for this type of reaction in the art, the process of the reaction can be monitored by means of a conventional test method in the art, and those skilled in the art can determine when to terminate the reaction according to monitoring results, so as to obtain a better reaction result. The present disclosure is preferably as follows.

In the salification reaction, the solvent may be an alcohol solvent (such as methanol). The compound represented by formula (A-1) and the compound represented by formula (B-1) are preferably in the form of aqueous solutions. The reaction temperature of the salification reaction may be room temperature (10 to 30° C.), and the reaction time may be 12 to 24 hours, for example, 16 hours. The following post-treatment steps may further be comprised after the completion of the salification reaction: extraction (e.g., extraction with chloroform) and concentration.

The above-mentioned method for preparing an onium salt may further comprise preparing a compound represented by formula (II) by means of the following steps:
step 1, in a solvent, reacting a compound represented by formula (III) with a compound represented by formula (IV) in the presence of an alkaline reagent; and
step 2, in a solvent, subjecting the reaction liquid obtained in step (1) to an oxidation reaction, so as to obtain the compound represented by formula (II);

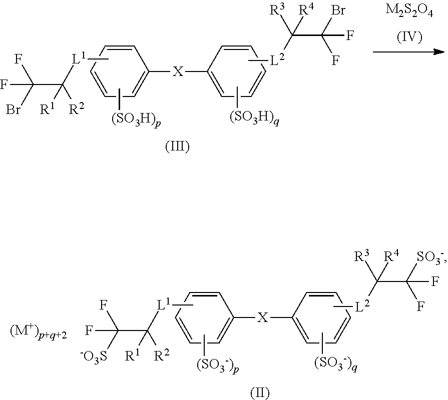

where the definitions of the various groups are as described in any solution of the present disclosure.

In step 1, the solvent may be a nitrile solvent (such as acetonitrile) and water. The alkaline reagent may be an alkali metal carbonate and/or an alkali metal bicarbonate, such as sodium bicarbonate. The reaction temperature of the reaction may be 40 to 80° C., for example, 70° C. The reaction time may be 12 to 24 hours, for example, 16 hours. The following post-treatment steps may further be comprised after the completion of the reaction: cooling (e.g., cooling to room temperature) and extraction (e.g., extraction with acetonitrile, prior to which a sodium chloride solid may be added to the reaction liquid until saturation).

In step 2, the solvent may be water. An oxidant for the oxidation reaction may be hydrogen peroxide. The reaction temperature of the oxidation reaction may be room temperature (10 to 30° C.). The reaction time may be 12 to 24 hours, for example, 16 hours. The following post-treatment steps may further be comprised after the completion of the oxidation reaction: extraction (e.g., extraction with acetonitrile), drying (e.g., drying with anhydrous sodium sulfate) and concentration.

The above-mentioned method for preparing an onium salt may further comprise preparing a compound represented by formula (III) by means of the following steps: in a solvent, subjecting a compound represented by formula (VI-1), a compound represented by formula (VI-2) and a compound represented by formula (V) to an esterification reaction as shown below, so as to obtain the compound represented by formula (III);

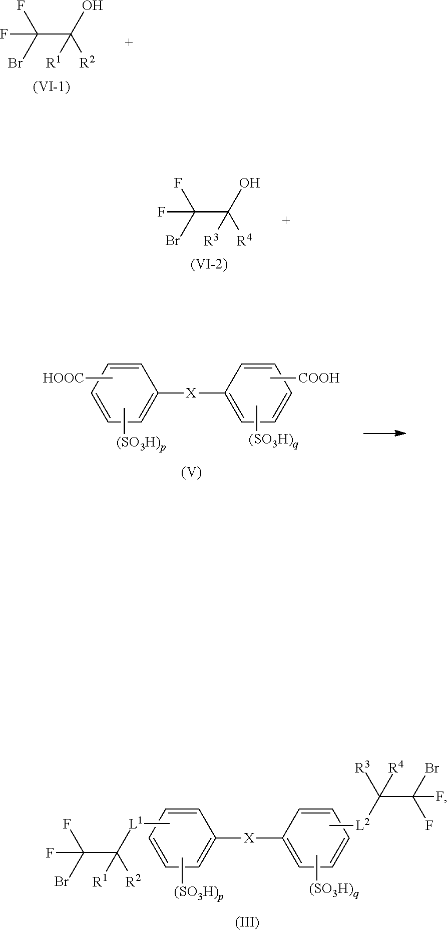

where the definitions of the various groups are as described in any solution of the present disclosure.

The esterification reaction may be carried out in the presence of a catalyst (such as toluenesulfonic acid). The solvent may be an aromatic hydrocarbon solvent (such as toluene). The temperature of the esterification reaction may be the refluxing temperature of the solvent. The reaction time of the esterification reaction may be 2 to 30 hours, for example, 8 hours.

The present disclosure further provides the use of the onium salt described above as a photoacid generator in a photoresist (e.g., a photoacid generator for dry a process by an ArF light source).

The present disclosure further provides a photoresist composition, which comprises the following components: the above-mentioned onium salt, a resin, an additive and an organic solvent.

In the photoresist composition, the types of the resin, the additive and the organic solvent may be of types that are conventionally used for photoresists in the art, and are preferably the following types in the present disclosure.

In one solution of the present disclosure, the resin in the photoresist composition may have a structure represented by (A)

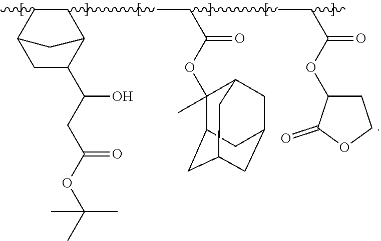

Preferably, the resin may be prepared by using the following preparation method: in the presence of an initiator (such as azodiisobutyronitrile, wherein the initiator may be 4 parts by mol relative to 100 parts by mol of the total reaction monomers), subjecting tert-butyl 3-bicyclo [2.2.1] hept-5-en-2-yl-3-hydroxypropionate, 1-adamantyl methacrylate and γ-butyrolactone acrylate (the molar ratio thereof may be 1:1: 1), which serve as monomers, to a polymerization reaction (e.g., a reaction at 65° C. for 16 hours) in a solvent (such as dioxane, wherein the solvent may be 300 parts by weight relative to 100 parts of by weight of the total reaction monomers), so as to obtain the resin (the weight-average molecular weight of the resin being 8000-9000 g/mol, for example, 8500 g/mol).

In one solution of the present disclosure, the additive in the photoresist composition may be a C₁₋₄ alkyl quaternary ammonium base, such as tetramethylammonium hydroxide.

In one solution of the present disclosure, the organic solvent in the photoresist composition may be an ester solvent, such as propylene glycol methyl ether acetate.

The contents of the onium salt, the resin, the additive and the organic solvent in the photoresist composition may be the contents conventionally used for photoresists in the art, and are preferably the following contents in the present disclosure.

In one solution of the present disclosure, the onium salt in the photoresist composition may be 2-10 parts by weight, for example, 4 parts by weight.

In one solution of the present disclosure, the resin in the photoresist composition may be 20-120 parts by weight, for example, 100 parts by weight.

In one solution of the present disclosure, the additive in the photoresist composition may be 0.1-1 part by weight, for example, 0.5 parts by weight.

In one solution of the present disclosure, the organic solvent in the photoresist composition may be 500-2000 parts by weight, for example, 1000 parts by weight.

In one solution of the present disclosure, the photoresist composition may comprise the following components in parts by weight: 4 parts by weight of the above-mentioned onium salt, 100 parts by weight of a resin, 0.5 parts by weight of an additive, and 1000 parts by weight of an organic solvent.

The present disclosure further provides a method for preparing the above-mentioned photoresist composition, the method comprising the following steps: mixing the above-mentioned components until uniform.

In the preparation method, the mixing may be carried out by a conventional mixing method in the art, preferably oscillation.

In the preparation method, the mixing step is preferably followed by filtration with a filter membrane, for example, filtration with a 0.2-μm filter membrane.

The present disclosure further provides the use of the above-mentioned photoresist composition in a photolithography process.

Herein, the photolithography process preferably comprises the following steps: coating a pretreated substrate with the photoresist composition, and subjecting same to drying (e.g., drying same at 110° C. for 90 seconds), exposure, and development (e.g., using an aqueous tetramethylammonium hydroxide solution as a developing solution).

On the basis of conforming to common knowledge in the art, the above-mentioned preferred conditions can be arbitrarily combined to obtain various preferred embodiments of the present disclosure.

Reagents and raw materials used in the present disclosure are all commercially available.

The positive effects of the present disclosure lie in: the photoresist comprising the onium salt provided in the present disclosure has better resolution, sensitivity and line width roughness.

### DETAILED DESCRIPTION OF THE PREFERRED EMBODIMENT

The present disclosure is further described below by way of examples; however, the present disclosure is not limited to the scope of the described examples. For the experimental methods in which no specific conditions are specified in the following examples, selections are made according to conventional methods and conditions or according to the product instructions.

### Preparation of Resin

In the examples or comparative examples of the disclosure, a resin was prepared according to the following method:

tert-butyl 3-bicyclo [2.2.1] hept-5-en-2-yl-3-hydroxypropionate (hereinafter referred to as BHP), 1-adamantyl methacrylate and y-butyrolactone acrylate were added at a molar ratio of 1:1:1. 1,4-dioxane was added as a polymerization solvent in an amount of 300 parts by weight relative to 100 parts by weight of the total amount of the reaction monomers, azodiisobutyronitrile was added as an initiator in an amount of 4 parts by mol relative to 100 parts by mol of the total amount of the reaction monomers, and the mixture was reacted at 65° C. for 16 hours.

After the reaction, the reaction solution was precipitated with n-hexane, and the resulting precipitate was removed and dried in vacuum. Therefore, a copolymer represented by the following formula was obtained, and the copolymer has a weight-average molecular weight of about 8500 g/mol.

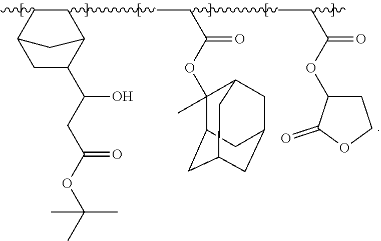

### Example 1

Photoacid generator 1 was synthesized with reference to the following synthesis route

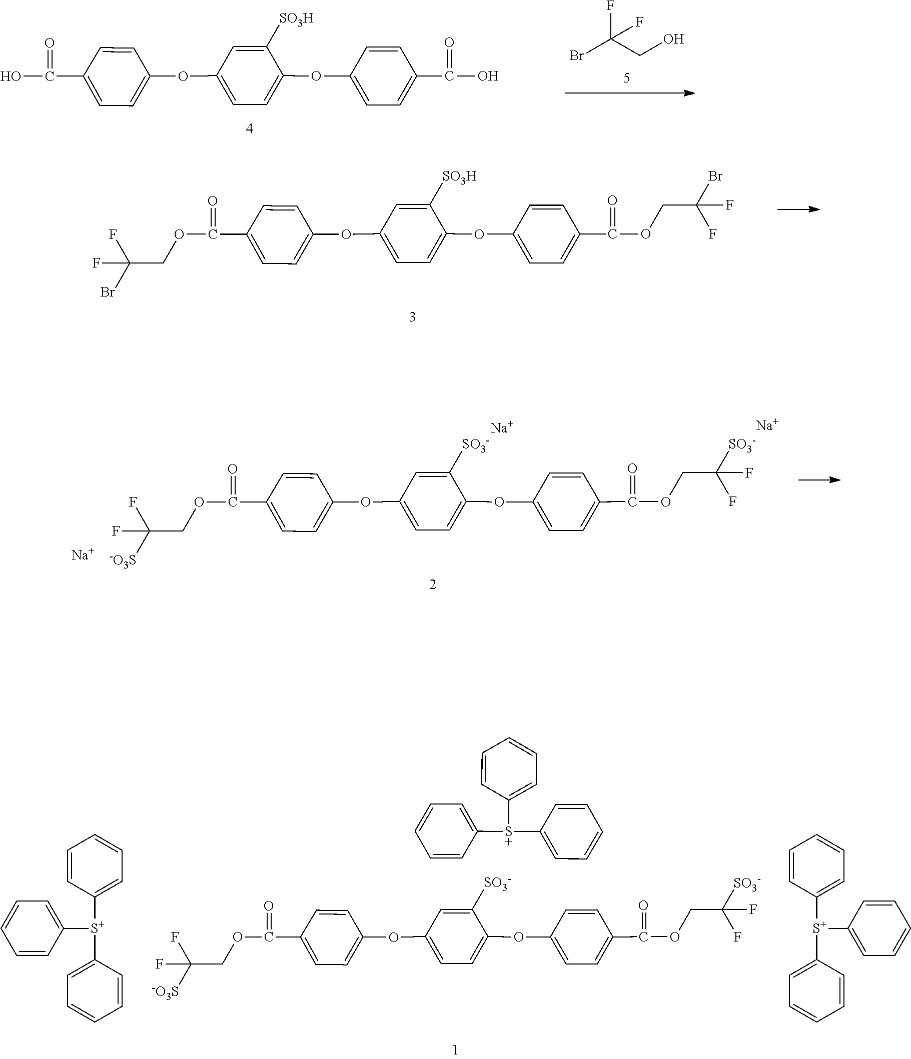

### Step 1: Synthesis of Compound 3

Compound 5 (12.07 g, 0.075 mol, 3.0 eq), compound 4 (10.76 g, 0.025 mol, 1.0 eq), p-toluenesulfonic acid (0.86 g, 0.005 mol, 0.2 eq) and 90 mL of toluene were added to a 250-mL glass bottle equipped with an oil-water separator and a condenser tube, and the resulting mixed liquid was heated and refluxed for 8 hours under stirring. Cooling was performed after the reaction was completed; the reaction liquid was washed with 50 mL of an aqueous sodium carbonate solution three times and with 50 mL of a saturated salt solution once; and the resulting organic phase was dried with anhydrous sodium sulfate and was then concentrated to obtain 11.9 g of compound 3 in total, with the yield being 66%.

LC-MS: 716.2

¹H NMR (300 MHz, DMSO): δ ppm: 5.07, 4H; 7.15, 4H; 7.31-7.68, 3H; 7.90, 4H; 8.5, 1H.

### Step 2: Synthesis of Compound 2

Compound 3 (10.7 g, 0.015 mol, 1.0 eq) was added to a 500-mL round bottom flask and dissolved, and 80 mL of acetonitrile was added thereto, followed by stirring to dissolve same. 80 mL of an aqueous solution containing sodium dithionite (5.2 g, 0.03 mol, 2.0 eq) and sodium bicarbonate (3.8 g, 0.045 mol, 3.0 eq) was dropwise added thereto in under the protection of nitrogen, and after the completion of the addition, the reaction liquid was heated and stirred at 70° C. for 16 hours. Then same was cooled, and a proper amount of a sodium chloride solid was added thereto until saturation. The reaction liquid was layered; the resulting aqueous phase was extracted twice with 30 mL of acetonitrile; and the resulting organic phases were combined and transferred to the 500-mL round bottom bottle, and 100 mL of pure water was added thereto. 30% hydrogen peroxide (3.6 g, 0.03 mol, 2.0 eq) was dropwise added to the mixed liquid under nitrogen, followed by stirring at room temperature for 16 hours. Layering was performed after the completion of the reaction; the resulting aqueous phase was extracted twice with 50 mL of acetonitrile; and the resulting organic phases were dried with anhydrous sodium sulfate, followed by concentration to obtain 7.6 g of compound 2 in total, with the yield being 64.6%.

¹H NMR (300 MHZ, DMSO): δ ppm: 5.13, 4H; 7.1, 4H; 7.31-7.68, 3H; 7.90, 4H.

### Step 3: Synthesis of Triphenylsulfonium Chloride

9.0 g of diphenyl sulfoxide and 60 mL of anhydrous dichloromethane were added to a 250-mL three-necked flask under the protection of nitrogen, and the reaction liquid was cooled 0° C. or less. The reaction liquid was maintained at 0° C. or less, and 14.5 g of trimethylchlorosilane was dropwise added. After the completion of the addition, the mixture was slowly heated to room temperature and further stirred for 1 hour. The reaction liquid was then cooled 0° C. or less again, and at the temperature, 67 mL of a 2 M solution of phenylmagnesium chloride in tetrahydrofuran was dropwise added thereto. After the completion of the addition, the mixture was slowly heated to room temperature and further stirred for 2 hours. The reaction liquid was then quenched with a small amount of water, and 75 mL of a 0.2 N aqueous hydrochloric acid solution was added thereto. After the mixed liquid was washed twice with 30 mL of ether, the resulting aqueous phase was an aqueous solution of triphenylsulfonium chloride, which was kept in a dark place for later use.

### Step 4: Synthesis of Compound 1

Compound 2 (7.5 g, 0.0096 mol, 1.0 eq) and 70 mL of methanol were added to a 250-mL round bottom flask, followed by stirring to dissolve same. The aqueous solution of triphenylsulfonium chloride (8.57 g, 0.0287 mol, 3.0 eq) prepared in advance was then dropwise added thereto under conditions without light. After the completion of the addition, stirring under conditions without light was continued for 16 hours. After the stirring was completed, the mixture was extracted with 30 mL of chloroform three times, and the resulting organic phases were combined and then washed twice with 30 mL of pure water. The mixture was layered, the resulting aqueous phase was removed, and the resulting organic phase was concentrated to obtain 12.2 g of compound 1 in total, with the yield being 84.6%.

¹H NMR (300 MHz, DMSO): δ ppm: 5.13, 4H; 7.15, 4H; 7.31, 1H; 7.33-7.36, 45H; 7.67, 2H; 7.90, 4H.

### Examples 2-6

Photoacid generators 2-6 of examples 2-6 were prepared with reference to example 1. The raw materials used, the products prepared in step 1 and the finally prepared photoacid generators are shown in tables 1, 2 and 3, respectively.

TABLE 1
Raw materials in examples 2-6

| No. | Raw material |
|---|---|
| 2 | 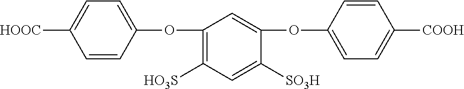 [SMILES: O=C(O)c1ccc(Oc2cc(Oc3ccc(C(=O)O)cc3)c(S(=O)(=O)O)cc2S(=O)(=O)O)cc1] |
| 3 | 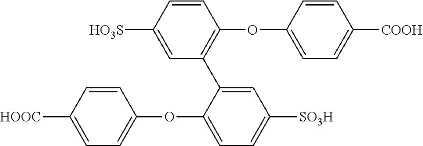 [SMILES: O=C(O)c1ccc(Oc2ccc(S(=O)(=O)O)cc2-c2cc(S(=O)(=O)O)ccc2Oc2ccc(C(=O)O)cc2)cc1] |
| 4 | 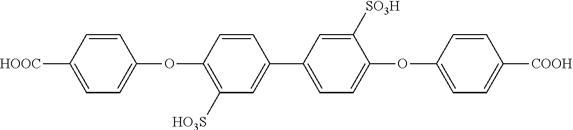 [SMILES: O=C(O)c1ccc(Oc2ccc(-c3ccc(Oc4ccc(C(=O)O)cc4)c(S(=O)(=O)O)c3)cc2S(=O)(=O)O)cc1] |
| 5 | 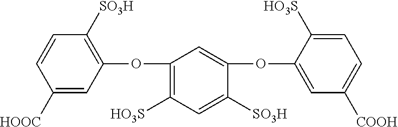 [SMILES: O=C(O)c1ccc(S(=O)(=O)O)c(Oc2cc(Oc3cc(C(=O)O)ccc3S(=O)(=O)O)c(S(=O)(=O)O)cc2S(=O)(=O)O)c1] |
| 6 | 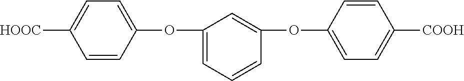 [SMILES: O=C(O)c1ccc(Oc2cccc(Oc3ccc(C(=O)O)cc3)c2)cc1] |

TABLE 2
Products prepared in step 1 in examples 2-6

| No. | Product prepared in step 1 |
|---|---|
| 2 | 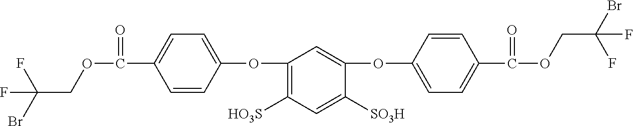 [SMILES: O=C(OCC(F)(F)Br)c1ccc(Oc2cc(Oc3ccc(C(=O)OCC(F)(F)Br)cc3)c(S(=O)(=O)O)cc2S(=O)(=O)O)cc1] |
| | ¹H NMR (300 MHz, DMSO): δ ppm: 5.07, 4H; 7.15, 4H; 7.35, 1H; 7.90, 4H; |
| | 8.42, 1H; 8.5, 2H. |
| 3 | 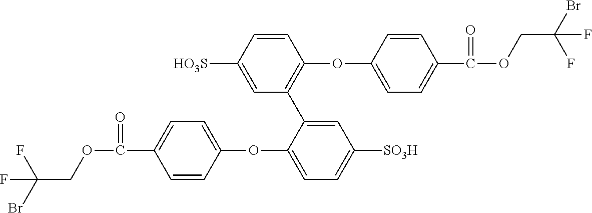 [SMILES: O=C(OCC(F)(F)Br)c1ccc(Oc2ccc(S(=O)(=O)O)cc2-c2cc(S(=O)(=O)O)ccc2Oc2ccc(C(=O)OCC(F)(F)Br)cc2)cc1] |
| | ¹H NMR (300 MHz, DMSO): δ ppm: 5.07, 4H; 7.15, 4H; 7.54, 2H; 7.86, 2H; |
| | 7.90, 4H; 8.24, 2H; 8.5, 2H. |
| 4 | 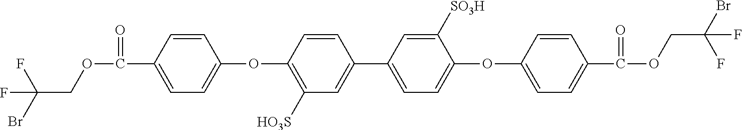 [SMILES: O=C(OCC(F)(F)Br)c1ccc(Oc2ccc(-c3ccc(Oc4ccc(C(=O)OCC(F)(F)Br)cc4)c(S(=O)(=O)O)c3)cc2S(=O)(=O)O)cc1] |
| | ¹H NMR (300 MHz, DMSO): δ ppm: 5.07, 4H; 7.15, 4H; 7.54, 2H; 7.90, 4H; |
| | 8.06-8.24, 4H; 8.5, 2H. |
| 5 | 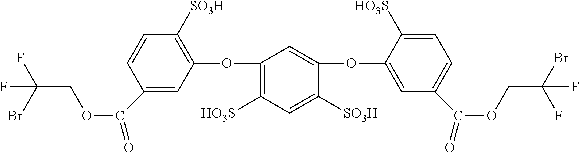 [SMILES: O=C(OCC(F)(F)Br)c1ccc(S(=O)(=O)O)c(Oc2cc(Oc3cc(C(=O)OCC(F)(F)Br)ccc3S(=O)(=O)O)c(S(=O)(=O)O)cc2S(=O)(=O)O)c1] |
| | ¹H NMR (300 MHz, DMSO): δ ppm: 5.07, 4H; 7.35, 1H; 7.67-7.90, 6H; 8.42, |
| | 1H; 8.5, 4H. |
| 6 | 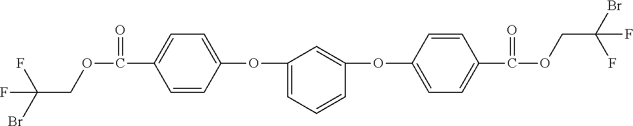 [SMILES: O=C(OCC(F)(F)Br)c1ccc(Oc2cccc(Oc3ccc(C(=O)OCC(F)(F)Br)cc3)c2)cc1] |
| | ¹H NMR (300 MHz, DMSO): δ ppm: 5.07, 4H; 6.64-6.52, 3H; 7.15, 4H; 7.3, |
| | 1H; 7.90, 4H. |

TABLE 3
Photoacid generators finally prepared in examples 2-6

| | | | Number |
|---|---|---|---|
| | | | of |
| | | | onium |
| No. | Anion | Onium ion | ions |
| 2 | 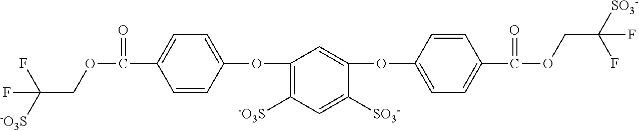 | 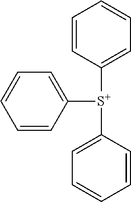 [SMILES: c1ccc([S+](c2ccccc2)c2ccccc2)cc1] | 4 |
| | ¹H NMR (300 MHz, DMSO): δ ppm: 5.07, 4H; 7.15, 4H; 7.33-7.36, 61H; 7.90, 4H; 8.42, 1H. | | |
| 3 | 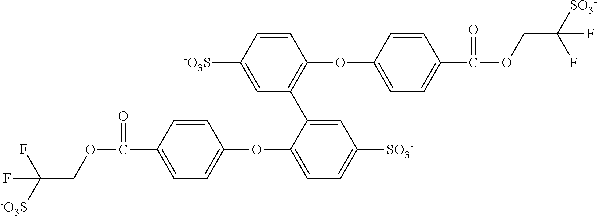 | 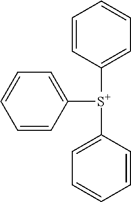 [SMILES: c1ccc([S+](c2ccccc2)c2ccccc2)cc1] | 4 |
| | ¹H NMR (300 MHz, DMSO): δ ppm: 5.07, 4H; 7.15, 4H; 7.33-7.36, 60H; 7.54, 2H; 7.86, 2H; 7.90, 4H; 8.24, 2H. | | |
| 4 | 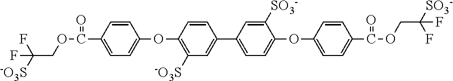 | 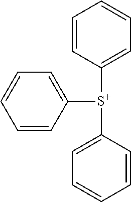 [SMILES: c1ccc([S+](c2ccccc2)c2ccccc2)cc1] | 4 |
| | ¹H NMR (300 MHz, DMSO): δ ppm: 5.07, 4H; 7.15, 4H; 7.33-7.36, 60H; 7.54, 2H; 7.90, 4H; 8.06-8.24, 4H. | | |
| 5 | 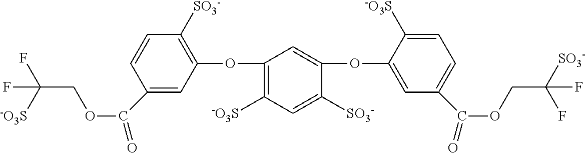 | 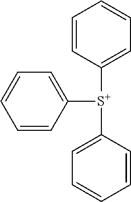 [SMILES: c1ccc([S+](c2ccccc2)c2ccccc2)cc1] | 6 |
| | ¹ HNMR (300 MHz, DMSO): δ ppm: 5.07, 4H; 7.35, 1H; 7.33-7.36, 90H; 7.67-7.90, 6H; 8.42, 1H. | | |
| 6 | 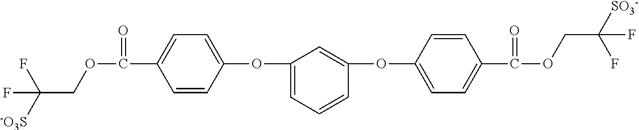 | 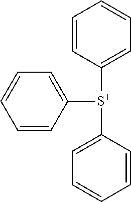 [SMILES: c1ccc([S+](c2ccccc2)c2ccccc2)cc1] | 2 |

¹H NMR (300 MHz, DMSO): δ ppm: 5.07, 4H; 6.64-6.52, 3H; 7.15, 4H; 7.3, 1H; 7.33-7.36, 30H; 7.90, 4H.

### Example 7: Preparation of Photoresist Composition and Comparative Photoresist Composition

The photoresist composition and comparative photoresist composition of the present disclosure were prepared according to the following method:

100 parts by weight of the resin prepared as above, 4 parts by weight of the photoacid generator prepared as above, and 0.5 parts by weight of tetramethylammonium hydroxide (as an alkaline additive) were dissolved in 1000 parts by weight of propylene glycol methyl ether acetate, and then the resulting solution was filtered by means of a 0.2-μm membrane filter, so as to prepare a photoresist composition. In such a case, photoacid generators in the photoresist compositions 1-6 and comparative photoresist compositions 1-15 are shown in Table 4.

TABLE 4

| No. of photoresist composition | Type of photoacid generator |
|---|---|
| Photoresist composition 1 | Photoacid generator 1 |
| Photoresist composition 2 | Photoacid generator 2 |
| Photoresist composition 3 | Photoacid generator 3 |
| Photoresist composition 4 | Photoacid generator 4 |
| Photoresist composition 5 | Photoacid generator 5 |
| Photoresist composition 6 | Photoacid generator 6 |
| Comparative photoresist | Comparative photoacid |
| composition 1 | generator 1 |
| Comparative photoresist | Comparative photoacid |
| composition 2 | generator 2 |
| Comparative photoresist | Comparative photoacid |
| composition 3 | generator 3 |
| Comparative photoresist | Comparative photoacid |
| composition 4 | generator 4 |
| Comparative photoresist | Comparative photoacid |
| composition 5 | generator 5 |
| Comparative photoresist | Comparative photoacid |
| composition 6 | generator 6 |
| Comparative photoresist | Comparative photoacid |
| composition 7 | generator 7 |
| Comparative photoresist | Comparative photoacid |
| composition 8 | generator 8 |
| Comparative photoresist | Comparative photoacid |
| composition 9 | generator 9 |
| Comparative photoresist | Comparative photoacid |
| composition 10 | generator 10 |
| Comparative photoresist | Comparative photoacid |
| composition 11 | generator 11 |
| Comparative photoresist | Comparative photoacid |
| composition 12 | generator 12 |
| Comparative photoresist | Comparative photoacid |
| composition 13 | generator 13 |
| Comparative photoresist | Comparative photoacid |
| composition 14 | generator 14 |
| Comparative photoresist | Comparative photoacid |
| composition 15 | generator 15 |

### Comparative photoacid generator 1: Bis(triphenylsulfonium salt) di(2-sulfo-2,2-difluoroethoxy) succinate

A preparation method for bis(triphenylsulfonium salt) di(2-sulfo-2,2-difluoroethoxy) succinate was the same as that in example 1.

| | | Number of |
|---|---|---|
| Anion | Onium ion | onium ions |
| 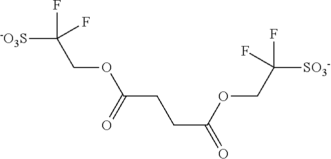 | 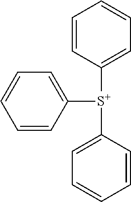 [SMILES: c1ccc([S+](c2ccccc2)c2ccccc2)cc1] | 2 |

¹H NMR (300 MHz, DMSO): δ ppm: 2.73, 4H; 4.90, 4H; 7.33-7.36, 30H.

### Comparative photoacid generators 2-15:

Comparative photoacid generators 2-15 were prepared according to example 1.

| Comparative | | | |
|---|---|---|---|
| photoacid | | | Number of |
| generator | Anion | Onium ion | onium ions |
| 2 | 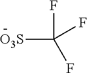 | 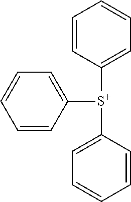 [SMILES: c1ccc([S+](c2ccccc2)c2ccccc2)cc1] | 1 |
| 3 | 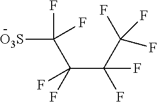 | 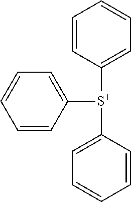 [SMILES: c1ccc([S+](c2ccccc2)c2ccccc2)cc1] | 1 |
| 4 | 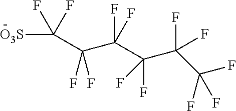 | 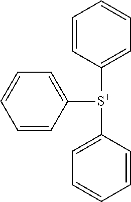 [SMILES: c1ccc([S+](c2ccccc2)c2ccccc2)cc1] | 1 |
| 5 | 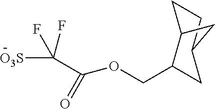 | 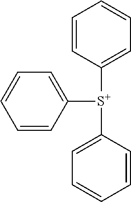 [SMILES: c1ccc([S+](c2ccccc2)c2ccccc2)cc1] | 1 |
| 6 | 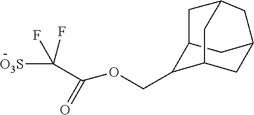 | 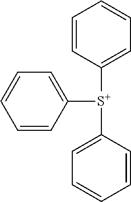 [SMILES: c1ccc([S+](c2ccccc2)c2ccccc2)cc1] | 1 |
| 7 | 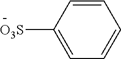 | 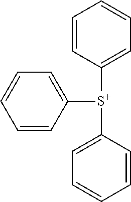 [SMILES: c1ccc([S+](c2ccccc2)c2ccccc2)cc1] | 1 |
| 8 | 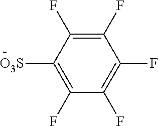 | 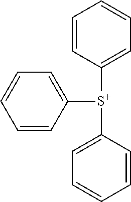 [SMILES: c1ccc([S+](c2ccccc2)c2ccccc2)cc1] | 1 |
| 9 | 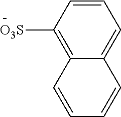 | 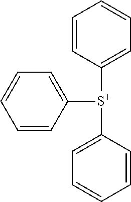 [SMILES: c1ccc([S+](c2ccccc2)c2ccccc2)cc1] | 1 |
| 10 | 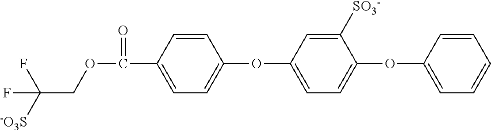 | 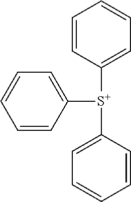 [SMILES: c1ccc([S+](c2ccccc2)c2ccccc2)cc1] | 2 |
| 11 | 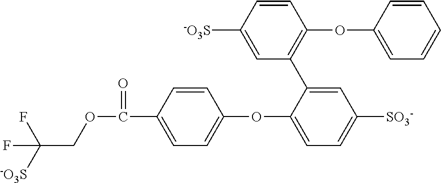 | 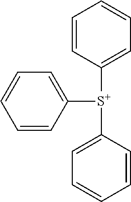 [SMILES: c1ccc([S+](c2ccccc2)c2ccccc2)cc1] | 3 |
| 12 | 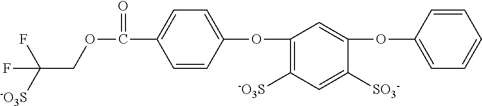 | 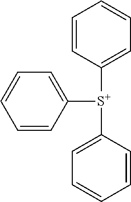 [SMILES: c1ccc([S+](c2ccccc2)c2ccccc2)cc1] | 3 |
| 13 | 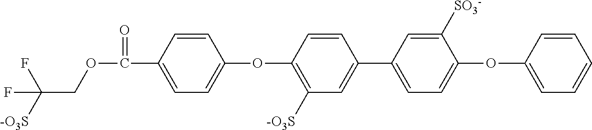 | 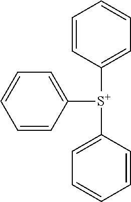 [SMILES: c1ccc([S+](c2ccccc2)c2ccccc2)cc1] | 3 |
| 14 | 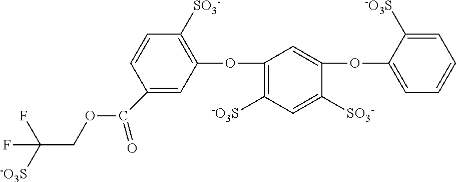 | 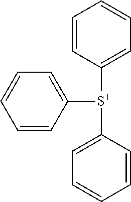 [SMILES: c1ccc([S+](c2ccccc2)c2ccccc2)cc1] | 5 |
| 15 | 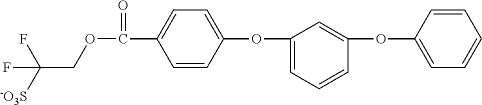 | 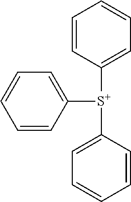 [SMILES: c1ccc([S+](c2ccccc2)c2ccccc2)cc1] | 1 |

### Application and Effect Embodiments

An anti-reflective coating of ARC-29 (Nissan Chemical Industries, Ltd.) was coated onto a silicon wafer (12 inches) using a spin coater and was then baked at 205° C. for 60 seconds to form a 70-nm-thick organic anti-reflective coating, and followed by coating with a prepared photoresist composition and drying at 110° C. for 90 seconds to form a film with a thickness of 0.20 μm. The resulting structure was subjected to exposure using a dry 193-nm exposure device (Nikon Corp., NA=0.68) and was baked at 105° C. for 60 seconds. The film was then developed with 2.38 wt % of an aqueous tetramethylammonium hydroxide solution for 40 seconds, then washed and dried. Therefore, a photoresist pattern was formed.

The exposure dose used for forming a 0.10-μm line-and-space (L/S) pattern at a line width of 1:1 after development was designated as the optimum exposure dose, and the optimum exposure dose was designated as the sensitivity (unit: mJ/cm²). The minimum pattern size resolved at this moment was specified as the resolution (unit: nm).

Moreover, in the case of line edge roughness (LER), the pattern roughness of the 0.10-μm line-and-space (L/S) pattern formed after development was observed, and the LER was measured (the smaller the value, the better the LER) (unit: nm).

The effects of photoresist compositions 1-6 and comparative photoresist compositions 1-15 are shown in Table 5.

TABLE 5

| No. of photoresist composition | Sensitivity | Resolution | LER |
|---|---|---|---|
| Photoresist composition 1 | 30 | 40 | 2.2 |
| Photoresist composition 2 | 32 | 50 | 3.2 |
| Photoresist composition 3 | 30 | 40 | 3.1 |
| Photoresist composition 4 | 35 | 60 | 3 |
| Photoresist composition 5 | 33 | 55 | 3.3 |
| Photoresist composition 6 | 40 | 65 | 3.5 |
| Comparative photoresist composition 1 | 55 | 70 | 6 |
| Comparative photoresist composition 2 | 53 | 69 | 8 |
| Comparative photoresist composition 3 | 56 | 69 | 5 |
| Comparative photoresist composition 4 | 58 | 73 | 7 |
| Comparative photoresist composition 5 | 54 | 68 | 5 |
| Comparative photoresist composition 6 | 55 | 71 | 8 |
| Comparative photoresist composition 7 | 59 | 69 | 8 |
| Comparative photoresist composition 8 | 58 | 70 | 7 |
| Comparative photoresist composition 9 | 53 | 72 | 5 |
| Comparative photoresist composition 10 | 51 | 68 | 7 |
| Comparative photoresist composition 11 | 55 | 69 | 4 |
| Comparative photoresist composition 12 | 58 | 72 | 5 |
| Comparative photoresist composition 13 | 57 | 71 | 7 |
| Comparative photoresist composition 14 | 59 | 71 | 6 |
| Comparative photoresist composition 15 | 51 | 69 | 5 |

What is claimed is:

1. An onium salt, wherein the onium salt has an anion and an onium ion, the anion having a structure represented by formula (I), the onium ion having a structure represented by formula (A) or formula (B), with the number of the onium ions keeping the charge of the onium salt neutral: 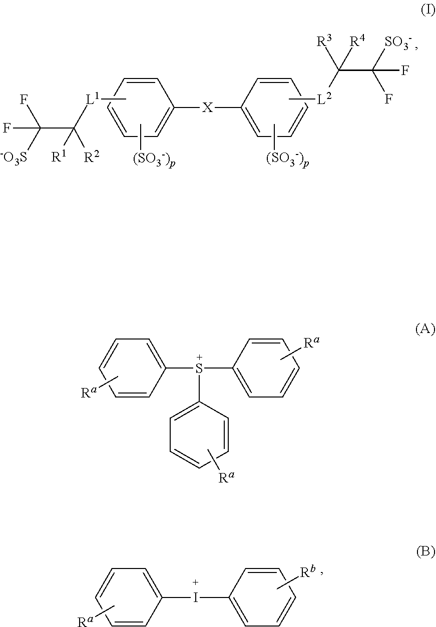
where R¹, R², R³ and R⁴ are each independently H or F; 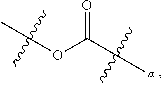 [SMILES: *OC(*)=O]
L¹ and L² are where the terminal a is connected to a benzene ring;
p and q are each independently 0, 1, 2, 3 or 4; 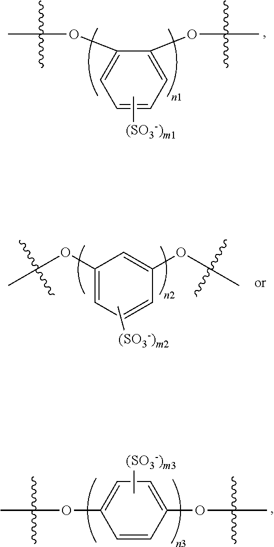
X is where
n1, n2 and n3 are each independently 1, 2, 3, 4 or 5;
m1, m2 and m3 are each independently 0, 1, 2, 3 or 4; and
R^{a} and R^{b} are each independently a halogen, a C₁₋₂₀ alkyl group or a C₁₋₂₀ alkoxy group, and the numbers of R^{a} and R^{b} are each independently 0 to 5.

2. The onium salt according to claim 1, wherein in the structure represented by formula (I),
R¹, R², R³ and R⁴ are the same;
or, p and q are the same;
or, the structural moiety 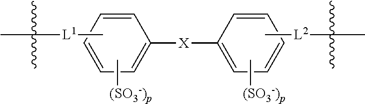 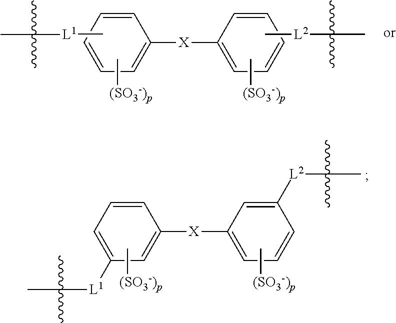
or, n1 is 2;
or, n2 is 1;
or, n3 is 1 or 2;
or, m1 is 1;
or, m2 is 0 or 2;
or, m3 is 1.

3. The onium salt according to claim 1, wherein in the structure represented by formula (I),
the structural moiety

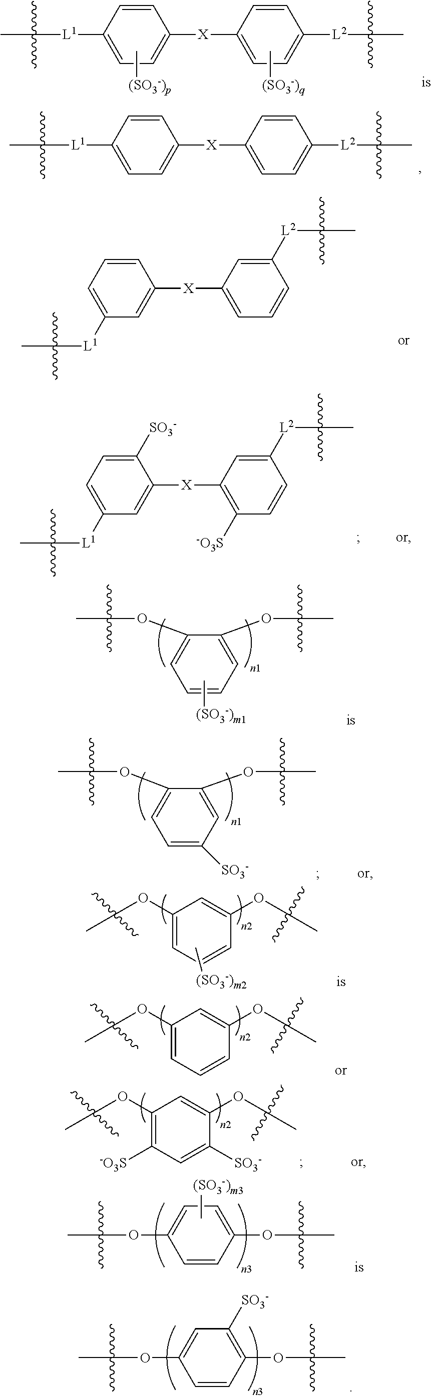

4. The onium salt according to claim 3, wherein in the structure represented by formula (I),

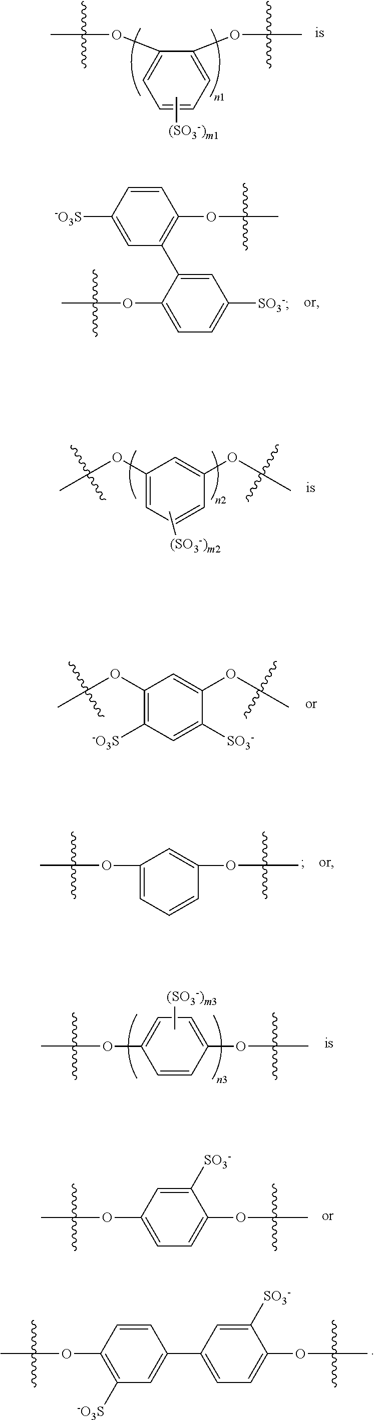

5. The onium salt according to claim 1, wherein the anion has a structure represented by formula (I-1), formula (I-2), formula (I-3) or formula (I-4),

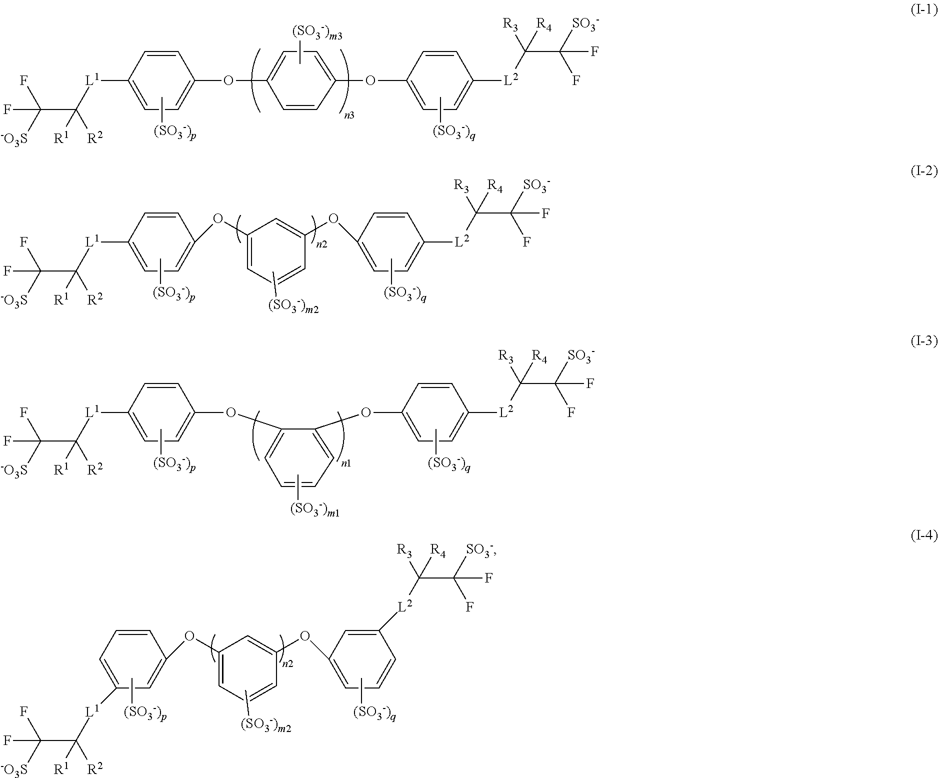

6. The onium salt according to claim 1, wherein in the structure represented by formula (A) or formula (B),
in the definitions of R^{a} and R^{b}, the halogens are each independently fluorine, chlorine, bromine or iodine;
or, in the definitions of R^{a} and R^{b}, the C₁₋₂₀ alkyl groups are each independently a methyl group, an ethyl group, a propyl group, an isopropyl group, an n-butyl group, an isobutyl group or a tert-butyl group;
or, in the definitions of R^{a} and R^{b}, the C₁₋₂₀ alkoxy groups are each independently such as a methoxy group, an ethoxy group, a propoxy group, an isopropoxy group, an n-butoxy group, an iso-butoxy group or a tert-butoxy group.

7. The onium salt according to claim 6, wherein the structure represented by formula (A) is: 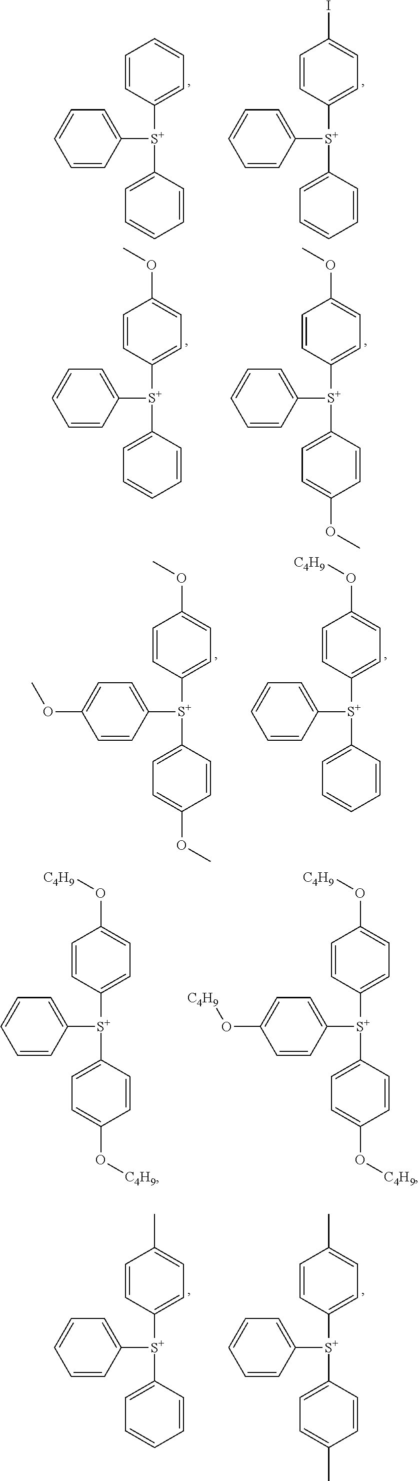 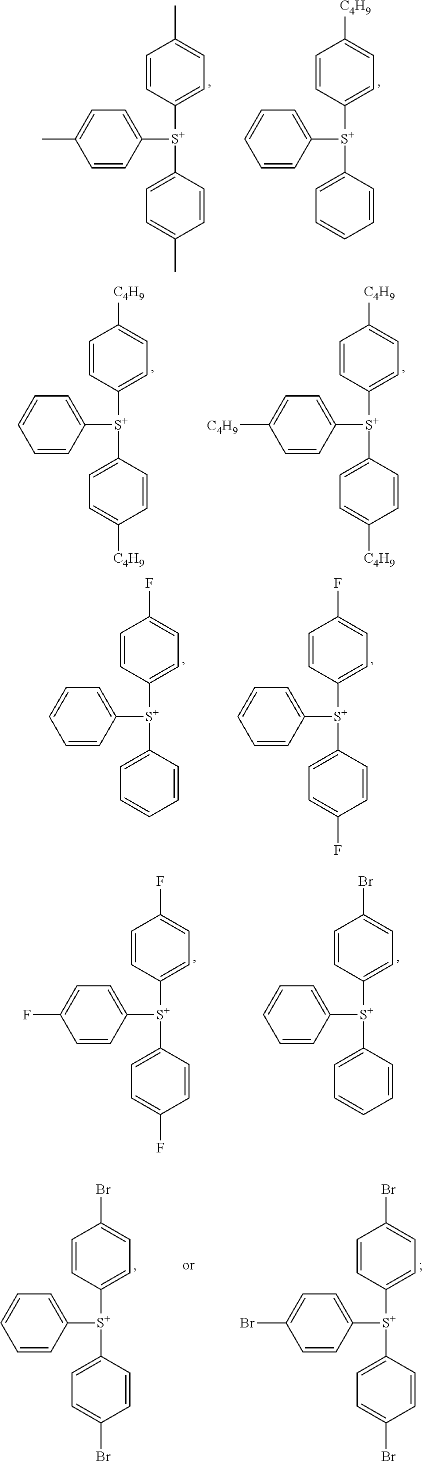
or, the structure represented by formula (B) is: 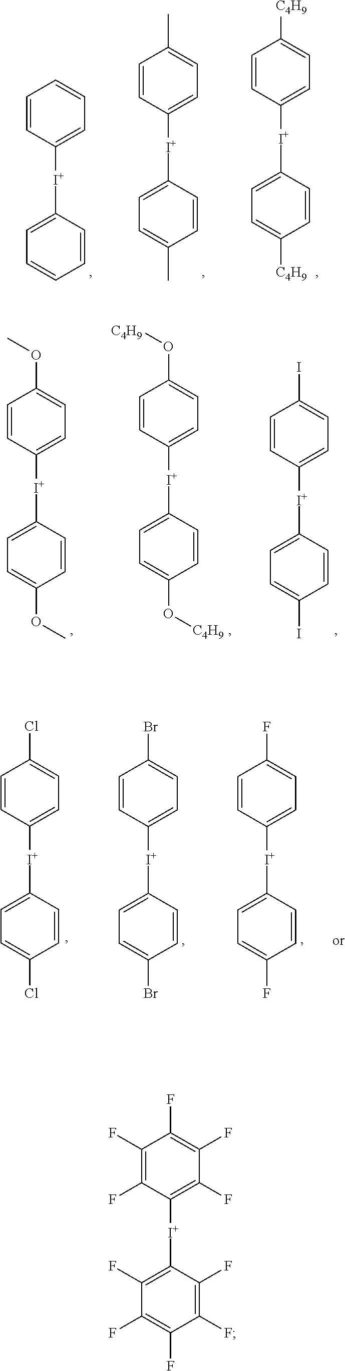
or, the anion is any one of the following structures:

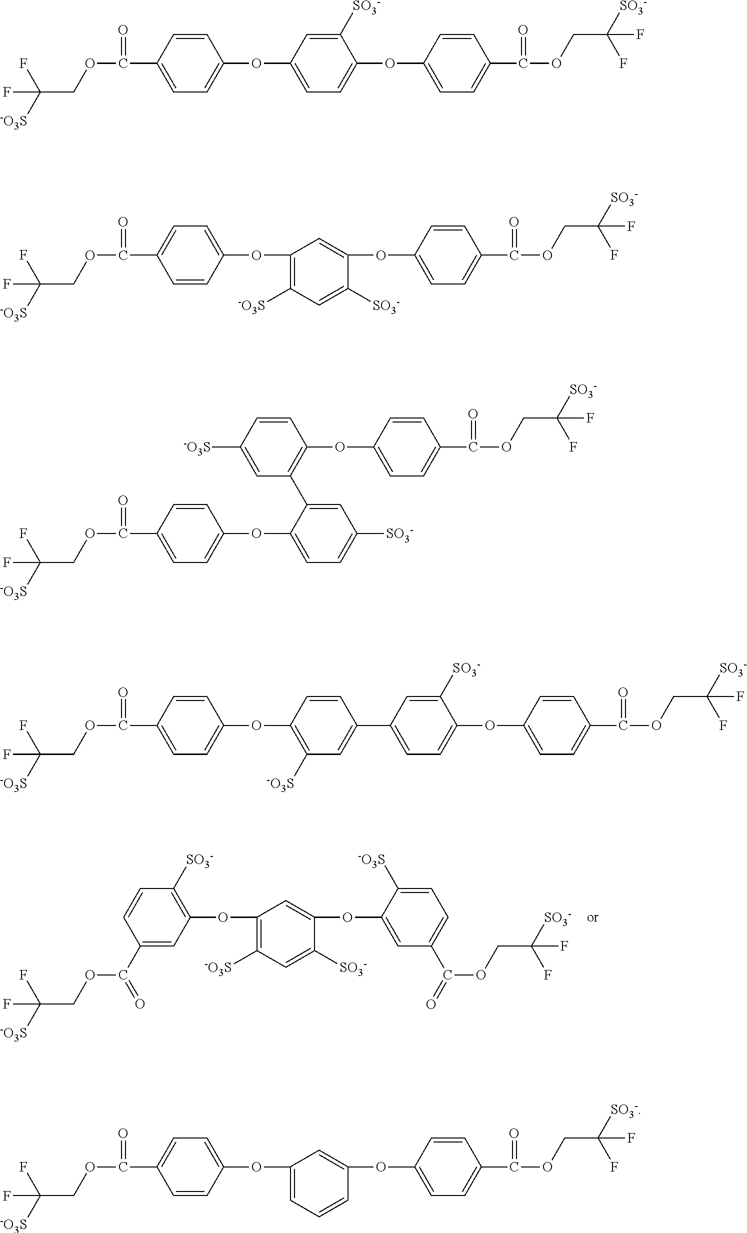

8-9. (canceled)

10. The onium salt according to claim 1, wherein the onium salt is any one of the following solutions:
solution 1: the anion is 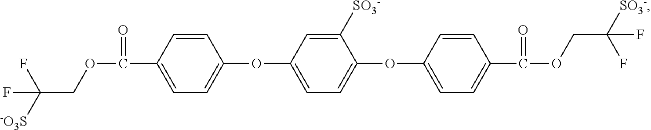
the onium ion is 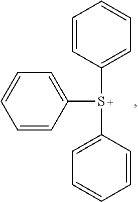
and the number of the onium ions is 3;
solution 2: the anion is 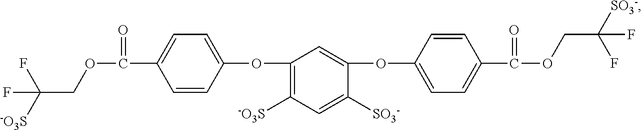
the onium ion is 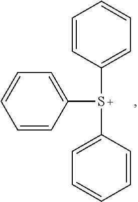
and the number of the onium ions is 4;
solution 3: the anion is 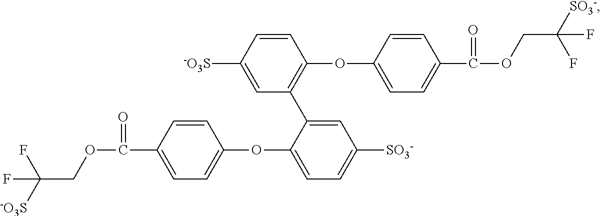
the onium ion is 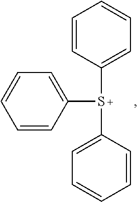
and the number of the onium ions is 4;
solution 4: the anion is 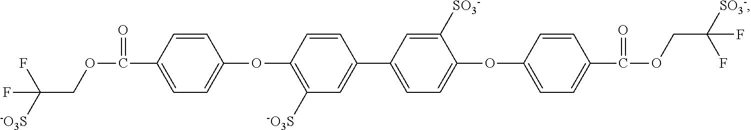
the onium ion is 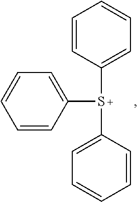
and the number of the onium ions is 4;
solution 5: the anion is 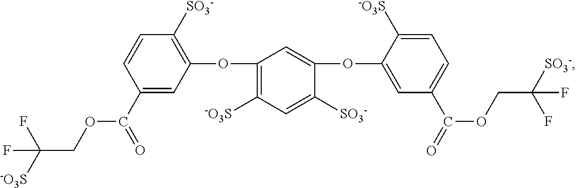
the onium ion is 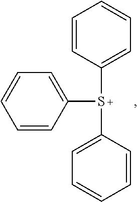
and the number of the onium ions is 6;
solution 6: the anion is 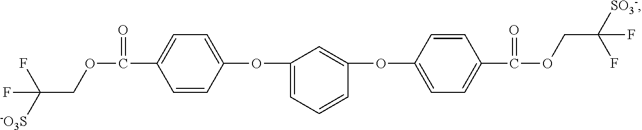
the onium ion is 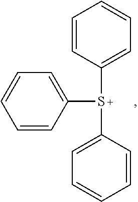
and the number of the onium ions is 2.

11. A method for preparing the onium salt according to claim 1, wherein the method comprises the following step: in a solvent, subjecting a compound represented by formula (II) and a compound represented by formula (A-1) or a compound represented by formula (B-1) to a salification reaction, so as to obtain the onium salt; 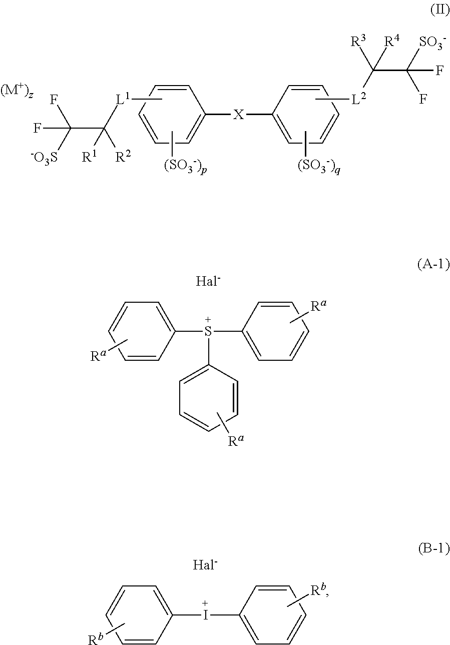
where M⁺ is Li⁺, Na⁺ or K⁺;
Ha⁻ is F⁻, Cl⁻, Br⁻ or I⁻;
z is (p+q+2+n1×m1), (p+q+2+n2×m2) or (p+q+2+n3 ×m3).

12. The method for preparing the onium salt according to claim 11, wherein the method meets one or more of the following conditions:
i. the solvent is an alcohol solvent;
ii. the compound represented by formula (A-1) and the compound represented by formula (B-1) are in the form of an aqueous solution;
iii. the reaction temperature of the salification reaction is room temperature;
iv. the reaction time of the salification reaction is 12 to 24 hours, for example, 16 hours.

13. The method for preparing the onium salt according to claim 11, wherein the method comprises preparing the compound represented by formula (II) by means of the following steps:
step 1, in a solvent, reacting a compound represented by formula (III) with a compound represented by formula (IV) in the presence of an alkaline reagent; and
step 2, in a solvent, subjecting the reaction liquid obtained in step (1) to an oxidation reaction, so as to obtain the compound represented by formula (II);

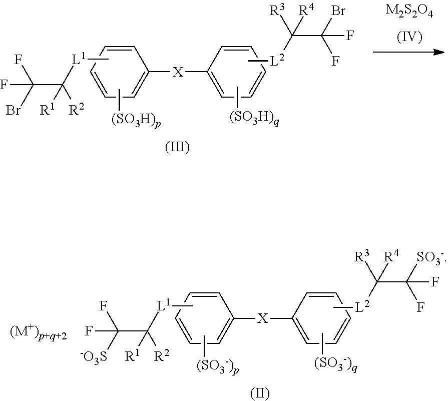

14. The method for preparing the onium salt according to claim 13, wherein the method meets one or more of the following conditions:
i. in step 1, the solvent is a nitrile solvent and water;
ii. in step 1, the alkaline reagent is an alkali metal carbonate or an alkali metal bicarbonate, such as sodium bicarbonate;
iii. in step 1, the reaction temperature of the reaction is 40 to 80° C.;
iv. in step 1, the reaction time of the reaction is 12 to 24 hours;
v. in step 2, the solvent is water;
vi. in step 2, an oxidant for the oxidation reaction is hydrogen peroxide;
vii. in step 2, the reaction temperature of the oxidation reaction is room temperature;
viii. in step 2, the reaction time of the oxidation reaction is 12 to 24 hours.

15. The method for preparing the onium salt according to claim 13, wherein the method comprises preparing the compound represented by formula (III) by means of the following steps: in a solvent, subjecting a compound represented by formula (VI-1), a compound represented by formula (VI-2) and a compound represented by formula (V) to an esterification reaction as shown below, so as to obtain the compound represented by formula (III); 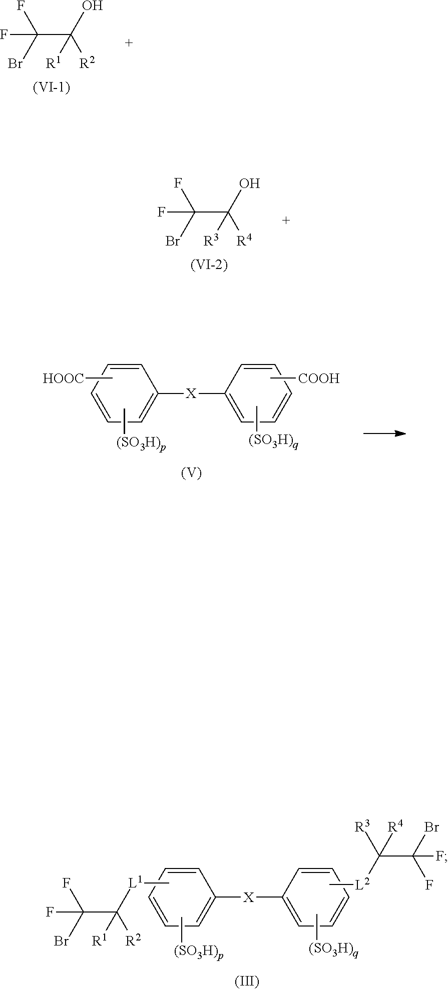
or, the method meets one or more of the following conditions:
i. in step 1, the solvent is acetonitrile and water;
ii. in step 1, the alkaline reagent is sodium bicarbonate;
iii. in step 1, the reaction temperature of the reaction is 70° C.;
iv. in step 1, the reaction time of the reaction is 16 hours;
v. in step 2, the reaction time of the oxidation reaction is 16 hours.

16. A photoacid generator in a photoresist, wherein the photoacid generator comprises the onium salt according to claim 1.

17. A photoresist composition, wherein the photoresist composition comprises the following components: the onium salt according to claim 1, a resin, an additive and an organic solvent.

18. The photoresist composition according to claim 17, wherein the resin has the following structural moiety 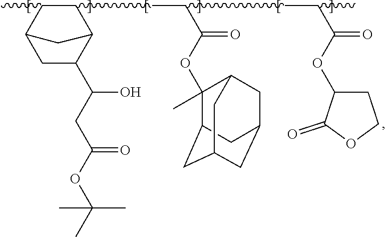
where the weight-average molecular weight of the resin is 8000-9000 g/mol;
or, the additive is a C₁₋₄ alkyl quaternary ammonium base;
or, the organic solvent is an ester solvent, such as propylene glycol methyl ether acetate;
or, in parts by weight, the onium salt accounts for 2-10 parts by weight;
or, in parts by weight, the resin accounts for 20-120 parts by weight;
or, in parts by weight, the additive accounts for 0.1-1 part by weight;
or, in parts by weight, the organic solvent accounts for 500-2000 parts by weight.

19. A photolithography process, wherein the photolithography process comprises the following steps: coating a pretreated substrate with the photoresist composition according to claim 17, and subjecting same to drying, exposure and development.

20. The method for preparing the onium salt according to claim 12, wherein the method meets one or more of the following conditions:
i. the solvent is methanol;
ii. the reaction time of the salification reaction is 16 hours.

21. The method for preparing the onium salt according to claim 15, wherein the method for preparing the compound represented by formula (III) meets one or more of the following conditions:
i. the esterification reaction is carried out in the presence of a catalyst, wherein the catalyst may be toluenesulfonic acid;
ii. the solvent is an aromatic hydrocarbon solvent;
iii. the temperature of the esterification reaction is the refluxing temperature of the solvent;
iv. the reaction time of the esterification reaction is 2 to 30 hours;

22. The method for preparing the onium salt according to claim 21, wherein the method for preparing the compound represented by formula (III) meets one or more of the following conditions:
i. the solvent is toluene;
ii. the reaction time of the esterification reaction is 8 hours.

23. The photoresist composition according to claim 18, wherein the additive is tetramethylammonium hydroxide;
or, the organic solvent is propylene glycol methyl ether acetate;
or, in parts by weight, the onium salt accounts for 4 parts by weight;
or, in parts by weight, the resin accounts for 100 parts by weight;
or, in parts by weight, the additive accounts for 0.5 parts by weight;
or, in parts by weight, the organic solvent accounts for 1000 parts by weight.
